(19) United States
(12) Patent Application Publication
(10) Pub. No.: US 2025/0268730 A1
(43) Pub. Date: Aug. 28, 2025

(54) BREAST PROSTHETIC DEVICE, FASTENING SYSTEM, AND USES THEREOF

(71) Applicant: Simonne C. CORMIER, Montague (CA)

(72) Inventor: Simonne C. CORMIER, Montague (CA)

(21) Appl. No.: 18/151,058

(22) Filed: Jan. 6, 2023

Related U.S. Application Data
(63) Continuation-in-part of application No. PCT/CA2021/050903, filed on Jun. 30, 2021.
(60) Provisional application No. 63/048,718, filed on Jul. 7, 2020.

(51) Int. Cl. A61F 2/52; A41C 3/14

(52) U.S. Cl. CPC A61F 2/52; A41C 3/148

(57) ABSTRACT

An prosthetic device is described for use as an artificial breast, comprising a shell and filler removably disposed within the shell, formed of non-synthetic material. The device is configured for compression fit within a clothing article, to inhibit movement of the artificial breast. The shell comprises a distal side defining a bra cup and a proximal side conforming to the wearer's body. The distal side of the shell comprises an inner felt layer forming the bra cup and an outer cloth layer facing the clothing article. The filler is removable from the shell, and provides form and firmness to the artificial breast, causing compression when worn. The shell flexibly accommodates different amounts of filler. The non-synthetic material has advantageous properties such as anti-bacterial, hypoallergenic, moisture wicking, biodegradable, breathable, and/or thermal regulating properties.

### CROSS-REFERENCE TO RELATED APPLICATIONS

This application is a continuation-in-part of and claims priority to International Patent Application No. PCT/CA2021/050903, filed Jun. 30, 2021, and claims the benefit of and priority to United States Provisional Patent Application number US 63/048,718, filed Jul. 7, 2020, the entire contents of which are hereby incorporated by reference.

### BACKGROUND OF THE INVENTION

The present disclosure relates generally to the fields of breast prosthetics and systems for securing such prosthetics in place.

Imagine going through surgery and, mere hours afterward, finding that what you thought would be the removal of a small lump would instead be far more expansive removal of tissue than expected. This is the reality for many individuals undergoing surgeries due to breast cancer. Whether the extent of tissue removal is unexpected or known in advance, such individuals move forward in their healing process in need of options for prosthetic devices that adequately address the discomfort of scarring, as well as the desired sizing and shaping.

Individuals may seek a prosthetic device that appears as similar to their pre-surgical size and shape as possible, while having a realistic feel and weight. Prosthetic devices may be used in addition to or as an alternative to reconstructive surgery.

Specialized bra suppliers have made efforts to address this need through individualized fittings. However, depending on the individual's preference or requirements, there may be limited options. Many such products involve a synthetic prosthesis fitted in a pocket within a specialty bra. Breast prostheses are offered by such companies as Amoena (Amoena Medizin-Orthopädie-Technik GmbH, Raubling, Germany), Anita (Anita Dr. Helbig GmbH, Brannenburg, Germany), and Jodee (Jodee, Hollywood, Florida, USA). There is a large market for prosthetic devices, and the cost of many such products can be covered by private medical insurance in many jurisdictions.

Available prostheses include silicone, polyester fill, or foam composite. Such products employ synthetic fibers or fabrics to form exterior shells, or insert pockets within a bra cup to hold such prostheses in place. Individuals who have undergone surgery for cancer may be wary of using products based on synthetic materials or fills, not only due to comfort considerations, but possibly because of concern over the possibility that toxins may be present in synthetic materials placed adjacent to the body.

U.S. Pat. No. 3,896,506 entitled “Breast Prosthesis” (issued Jul. 29, 1975), describes a gel-filled prosthesis to be worn inside of a specialized breast cup of a bra. Similarly, U.S. Pat. No. 3,911,503 entitled “Breast Prosthesis” (issued Oct. 14, 1975), describes a silicone rubber prosthesis to be worn inside of a specialized breast cup of a bra. U.S. Pat. No. 3,896,506 entitled “Breast Prosthesis” (issued Jul. 29, 1975), describes a gel-filled prosthesis to be worn inside of a specialized breast cup of a bra. U.S. Pat. No. 5,133,752 entitled “Launderable Prosthetic Device” (issued Jul. 28, 1992), describes a breast prosthesis formed of porous layers, such as a polyester foam. U.S. Pat. No. 6,432,138 entitled “Controlled Porosity 3-D Fabric Breast Prosthesis” (issued Aug. 13, 2002), describes a non-biodegradable breast prosthesis filled with monofilament polyester.

There is a need for further innovation, to provide comfortable breast

prosthesis products with a realistic look and feel, which also satisfy an individual's requirements or preferences for comfort and reduced exposure to toxins that may be present in the products' synthetic materials.

### SUMMARY

A breast prosthesis device is described herein which addresses at least one deficiency of at least one previously known breast prosthesis product or device. Further, an optional system for removably fastening a breast prosthesis in position in an article of clothing is described. A variety of uses of the described device and fastening system are also described.

There is described herein an artificial breast comprising a shell and a filler removably disposed within the shell, said artificial breast comprising non-synthetic material; said artificial breast being shaped and configured to fit securely within a clothing article by compression between a wearer and the clothing article to inhibit movement of the artificial breast relative to the clothing article; wherein: the shell comprises a distal side defining a curved bra cup and a proximal side configured to conform to contours of a body and configured to expand, the distal side of the shell comprising an inner felt layer configured to form the bra cup and an outer cloth layer for facing the clothing article when worn, and the filler is removably disposed within the shell in an amount configured to provide form and firmness to the artificial breast and in an amount adequate to cause compression of the artificial breast when worn; wherein the proximal side of the shell defines a flexible opening for disposing the filler within the shell, and said shell flexibly accommodates different amounts of filler.

Additionally, described herein is a prosthetic device comprising an artificial breast comprising a shell and a filler removably disposed within the shell, said artificial breast comprising non-synthetic material; at least two fastening straps coupled to the artificial breast; and at least two loops configured to couple to a clothing article; wherein each of the at least two fastening straps is configured to releasably fasten to each of the at least two loops, respectively, to secure the artificial breast to the clothing article and inhibit movement of the artificial breast relative to the clothing article.

Further, described herein is a fastening system comprising a fastening strap configured to couple to a first article, the fastening strap comprising a fastener disposed thereon; and a loop configured to be coupled to a second article; the fastening strap being configured to releasably fasten to the loop to secure the first article to the second article.

Described herein is a method of securing the prosthetic device to a clothing article comprising an inner shelf, the method comprising coupling the at least two loops to the inner shelf of the clothing article; and fastening the at least two fastening straps to the respective loops, thereby securing the artificial breast to the inner shelf of the clothing article.

Further, described herein is a kit, comprising an artificial breast comprising a shell and a filler removably disposed within the shell, said artificial breast comprising non-synthetic material; at least two fastening straps configured to couple to the artificial breast; at least two loops configured to couple to a clothing article; each of the at least two fastening straps being configured to releasably fasten to each of the at least two loops, respectively, to secure the artificial breast to the clothing article and inhibit movement of the artificial breast relative to the clothing article; and instructions for installation and use.

Described herein is a clothing article comprising the prosthetic device, wherein the artificial breast is releasably fastenable to the clothing article; and use of the prosthetic device in a clothing article. Further, described herein is a bra comprising the prosthetic device.

Further, described herein is a feed tab comprising a body having a first end and a second end; the first end configured to couple to a fastening strap for coupling to a first article, and the second end comprising a tapered tab configured to feed the feed tab through a loop for coupling to a second article.

Described herein is a loop strip, comprising a loop coupled to a base, the loop configured to receive a fastening strap coupled to a first article, the base having a first side to which the loop is coupled and a second side configured to couple to a second article.

Advantageously, according to certain embodiments described herein, the use of non-synthetic materials in the described device results in anti-bacterial properties, hypoallergenic properties, moisture wicking ability, biodegradability, breathability, and/or thermoregulatory improvements as compared with previous technologies.

An advantage of embodiments pertaining to the described fastening strap and loop system is that a prosthetic device may be well-secured in a bra, such as a sports bra, or other articles of clothing whereas previous technologies may have resulted in a poorly secured prosthesis.

### BRIEF DESCRIPTION OF THE FIGURES

Embodiments of the present disclosure will now be described, by way of example only, with reference to the attached Figures.

FIG. 1 schematically depicts, in section, the layers within the artificial breast, including filler, inner cloth layer, felt layer, and outer cloth layer.

FIG. 2 depicts general steps 1 and 2 in a process for forming an embodiment of the artificial breast according to Example 1.

FIG. 3 depicts general steps 3 and 4 in a process for forming an embodiment of the artificial breast according to Example 1.

FIG. 4 depicts general step 5 in a process for forming the artificial breast according to Example 1, together with representative packaging.

FIG. 5 depicts a ribbon having multiple loops disposed thereon (loop strip) for placement within a clothing article.

FIG. 6 depicts a fastening strap with a rounded feed tab.

FIG. 7 depicts an embodiment of a snap cap having an integral tapered and curved feed tab.

FIG. 8 depicts options for placement of loops in a bra.

FIG. 9 depicts a fastening strap during and after fastening to a loop.

FIG. 10 depicts an embodiment of the artificial breast before and after fastening to the loops in a clothing article

FIG. 11 depicts a different configurations (a) to (j) for ribbons having one or more loop disposed thereon (loop strips) for placement within a clothing article.

FIG. 12 depicts an inverted version of the artificial breast in, as shown in

FIG. 4, for an embodiment having no requirement for a fastener or fastening system.

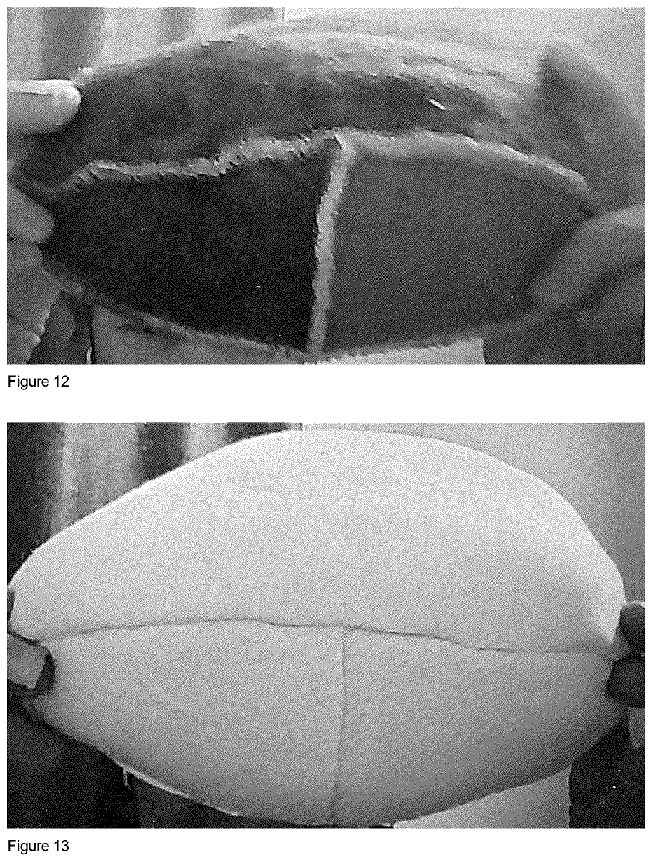

FIG. 13 depicts the non-inverted finished article of an artificial breast as shown in FIGS. 4 and 12.

### DETAILED DESCRIPTION

The breast prosthetic device, fastening system, feed tab, loop strip, and uses described herein serve to meet one or more of the unmet needs in the field. The described device secures an artificial breast in place in respect of an article of clothing while inhibiting movement of the artificial breast, relative to the clothing. The desired secure positioning can be accomplished no matter how active the wearer is.

Comfort and security against unwanted movement builds the confidence of the wearer. Advantageously, the use of non-synthetic materials, in particular in body-facing regions of the device, reduces exposure of the wearer to synthetic materials, which may allay concerns regarding toxins and/or chemicals presumed to be associated with synthetic materials. By providing a device based on natural fibers and fabrics, individuals concerned about synthetic materials from a health perspective can set this concern aside. Such non-synthetic materials may possess additional advantages, such as being environmentally friendly, biodegradable (thereby returning to nature) following eventual disposal, derived from renewable resources, softer feeling, natural thermal regulation, anti-bacterial properties, hypoallergenic properties, breathability, and/or moisture wicking tendencies are also observed in many natural materials. The durability of natural materials, and renewal of shape after washing may also exceed that of many synthetic materials.

The device is appropriate for use not only by breast cancer survivors, but also for individuals wishing to achieve breast enhancement, to correct asymmetric breasts, or for transgender persons.

The device is referenced herein interechangeably as an “artificial breast”. The artificial breast comprises a shell and a filler removably disposed within the shell, the artificial breast comprising non-synthetic material, and being shaped and configured to fit securely within a clothing article by compression between a wearer and the clothing article to inhibit movement of the artificial breast relative to the clothing article. The shell comprises a distal side defining a curved bra cup and a proximal side configured to conform to contours of a body and configured to expand, the distal side of the shell comprising an inner felt layer configured to form the bra cup and an outer cloth layer for facing the clothing article when worn. The filler is removably disposed within the shell in an amount configured to provide form and firmness to the artificial breast and in an amount adequate to cause compression of the artificial breast when worn. The proximal side of the shell defines a flexible opening for disposing the filler within the shell, and said shell flexibly accommodates different amounts of filler.

The non-synthetic material has anti-bacterial, hypoallergenic, moisture wicking, biodegradable, breathable, or thermal regulating properties, or a combination thereof. The felt layer of the shell may comprise felted Alpaca fiber, wool fiber, rayon fiber, or a combination thereof; and the filler may comprise Alpaca fiber, hemp fiber, wool fiber, cotton fiber, bamboo fiber, silk fiber, rayon fiber, or a combination thereof. Further, the shell may additionally comprise an inner cloth layer comprises Alpaca fiber, hemp fiber, wool fiber, cotton fiber, bamboo fiber, silk fiber, rayon fiber, or a combination thereof. An exemplary filler is Alpaca fiber, and an exemplary inner felt layer of the shell comprises felted Alpaca fiber. An exemplary fiber for the proximal side of the shell comprises bamboo and/or cotton.

The proximal side of the shell may be configured to expand relative to the amount of filler disposed within the shell to conform the proximal side of the shell to the contours of a body.

The distal side of the shell may be formed as a horizontally approximately oblong shape comprising an upper portion configured as a semi-oblong, and two lower oblong quadrants forming the lower portion of the oblong shape with an approximately central vertical seam between the quadrants to define the contours of the bra cup. The upper portion of the oblong shape of the distal side of the shell may be configured to the wearer's desired shape and volume of bra cup. The upper portion and the two lower portions of the oblong shape of the distal side of the shell may be each configured to the wearer's desired shape and volume of bra cup.

The bra cup size and shape can be individually adjusted or tailored to suit to the needs or desires of the wearer. For example, the wearer may be one who has had a full mastectomy, a partial mastectomy, and/or who wishes to supplement existing breast volume.

In one exemplary embodiment, the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn.

A weight may be disposed within the shell, such as for example a porous rock.

The clothing article within which the artificial breast is to be worn may comprise a top, a bra, a top, a dress, lingerie, a bralette, or a swimsuit.

The artificial breast may optionally comprise at least two fastening straps for securing the artificial breast within a clothing article having loops configured to receive the fastening straps, wherein resaid fastening straps comprise a ribbon having a first end and a second end, the first end coupling to the shell of the artificial breast; and the second end comprising a feed tab and a fastener, the feed tab being configured for feeding the second end of each of the fastening straps through the respective loop of the clothing article.

Optionally, the fastener is a snap, a hook and loop fastener, or an adhesive fastener.

For the configuration in which fasteners are used, the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn. Further, in this configuration, the distal side of the shell may be formed as a horizontally approximately oblong shape comprising an upper portion configured as a semi-oblong, and two lower oblong quadrants forming the lower portion of the oblong shape with an approximately central vertical seam between the quadrants to define the contours of the bra cup.

In other aspects of the invention described herein, a prosthetic device is described which is fastened in place. A prosthetic device is described herein, the device comprising an artificial breast comprising a shell and a filler removably disposed within the shell, said artificial breast comprising non-synthetic material; at least two fastening straps coupled to the artificial breast; and at least two loops configured to couple to a clothing article; wherein each of the at least two fastening straps is configured to releasably fasten to each of the at least two loops, respectively, to secure the artificial breast to the clothing article and inhibit movement of the artificial breast relative to the clothing article.

The non-synthetic material of the artificial breast can have anti-bacterial, hypoallergenic, moisture wicking, biodegradable, breathable, or thermal regulating properties, or a combination thereof.

The shell of the artificial breast can comprise a distal side defining a bra cup and a proximal side configured to conform to contours of a body; and the filler of the artificial breast can be removably disposed within the shell in an amount configured to provide form and firmness to the artificial breast. The filler can comprise Alpaca fiber, hemp fiber, wool fiber, cotton fiber, bamboo fiber, silk fiber, rayon fiber, or a combination thereof. The distal side of the shell can comprise a felt layer, the felt layer being configured to form the bra cup. The felt layer can comprise Alpaca fiber, wool fiber, rayon fiber, or a combination thereof. The distal side of the shell can further comprise an inner and an outer cloth layer, the felt layer being disposed therebetween. The inner cloth layer can comprise Alpaca fiber, hemp fiber, wool fiber, cotton fiber, bamboo fiber, silk fiber, rayon fiber, or a combination thereof. The proximal side of the shell can comprise a flexible layer, the flexible layer being configured to expand relative to the amount of filler disposed within the shell to conform the proximal side of the shell to the contours of a body. The flexible layer can define an opening for disposing the filler within the shell. The flexible layer can comprise Alpaca fiber, hemp fiber, wool fiber, cotton fiber, bamboo fiber, silk fiber, rayon fiber, or a combination thereof.

Each of the at least two fastening straps coupled to the artificial breast can comprise a ribbon having a first end and a second end, the first end coupling to the artificial breast; and the second end comprising a feed tab, the feed tab being configured for feeding the second end of each of the fastening straps through the respective loop. The feed tab can be rigid or resilient. The feed tab can be upwardly curved relative to the fastening strap to conform with the contour of the artificial breast. The second end of each of the at least two fastening straps can further comprise a fastener for coupling each of the at least two fastening straps through the respective loop. The fastener can be a snap, a hook and loop fastener, or an adhesive fastener.

Each of the at least two loops may be coupled to a base, where the base may have a first side to which a loop is coupled and a second side configured to couple to the clothing article. The base may also be flexible. The loop coupled to the base may be substantially centered on the base, or off-centered. Each of the at least two loops may be part of a loop strip, where the loop strip comprises the at least two loops and a base. The base of the loop strip may have a first side to which the at least two loops are coupled, and a second side configured to couple to the clothing article. The base of the loop strip may also be flexible. The at least two loops may be substantially aligned and centered along a length of the first side of the base of the loop strip, or may be off-centered. The base of the loop strip may comprise a perforation guide for indicating where to cut the loop strip. The perforation guide may be a visual or partial perforation guide.

The artificial breast of the device can further comprise a weight disposed within the shell. The weight can be a porous rock.

The device can comprise at least three fastening straps coupled to the artificial breast, and at least three loops configured to couple to the clothing article.

A fastening system is also described herein, the fastening system comprising a fastening strap configured to couple to a first article, the fastening strap comprising a fastener disposed thereon; and a loop configured to be coupled to a second article; the fastening strap being configured to releasably fasten to the loop to secure the first article to the second article. The fastening strap can comprise a flexible ribbon, said fastening strap having a first end and a second end, wherein the first end is configured for coupling to the first article; and the second end comprises a feed tab, the feed tab being configured to feed the second end of the fastening strap through the loop. The feed tab can be rigid or resilient. The feed tab can be curved upwardly from the fastening strap. The fastener can be disposed at the second end of the fastening strap. The fastener can be a two part cooperating fastener, with the two parts disposed in sequence at the second end of the fastening strap. The fastener can be a snap, a hook and loop fastener, or an adhesive fastener. The fastener can be a snap and the feed tab can be integral to the snap.

The loop of the fastening system can be coupled to a base, where the base may have a first side to which the loop is coupled, and a second side configured to couple to the second article. The base may also be flexible. The loop coupled to the base may be substantially centered on the base, or may be off-centered. The loop of the fastening system may be part of a loop strip comprising at least two loops and a base. The base of the loop strip may have a first side to which the at least two loops are coupled and a second side configured to couple to the second article. The base of the loop strip may also be flexible. The at least two loops of the loop strip may be substantially aligned and centered along a length of the first side of the base, or may be off-centered. The base of the loop strip may comprise a perforation guide for indicating where to cut the loop strip. The perforation guide may be a visual or partial perforation guide.

Also described is a method of securing the prosthetic device as described herein to a clothing article comprising an inner shelf, the method comprising coupling the at least two loops to the inner shelf of the clothing article; and fastening the at least two fastening straps to the respective loops, thereby securing the artificial breast to the inner shelf of the clothing article.

Further, there is described herein a kit, comprising an artificial breast comprising a shell and a filler removably disposed within the shell, said artificial breast comprising non-synthetic material; at least two fastening straps configured to couple to the artificial breast; at least two loops configured to couple to a clothing article; each of the at least two fastening straps being configured to releasably fasten to each of the at least two loops, respectively, to secure the artificial breast to the clothing article and inhibit movement of the artificial breast relative to the clothing article; and instructions for installation and use.

A clothing article is described herein, the clothing article comprising the prosthetic device as described herein, wherein the artificial breast is releasably fastenable to the clothing article. The clothing article can comprise an inner shelf and the loops are coupled to the clothing article along the inner shelf. The clothing article can be a top, a bra, a dress, lingerie, a bralette, or a swimsuit.

A bra is also described herein, the bra comprising the prosthetic device as described herein. The bra can comprise a front closure.

Further, described is a use of the prosthetic device as described herein in a clothing article. The clothing article can be a top, a bra, a dress, a lingerie article, a bralette, or a swimsuit. The clothing article can comprise an inner shelf. The clothing article can be a bra.

A feed tab is described herein, the feed tab comprising a body having a first end and a second end; the first end configured to couple to a fastening strap for coupling to a first article, and the second end comprising a tapered tab configured to feed the feed tab through a loop for coupling to a second article. The tapered tab can be rigid or resilient. The tapered tab can be curved upwardly from the body. The body can be rigid or resilient. The first end of the body can be further configured to couple to, or be integral to a first part of a two-part cooperating fastener for releasably fastening the strap to the loop to secure the first article to the second article when the strap comprises a second part of the cooperating fastener. The two-part cooperating fastener can be a snap, a hook and loop fastener, or an adhesive fastener. The fastener can be a snap and the feed tab can be integral to the snap.

Also described herein is a loop strip, the loop strip comprising a loop coupled to a base, the loop configured to receive a fastening strap coupled to a first article, the base having a first side to which the loop is coupled and a second side configured to couple to a second article. The loop can be substantially centered on the base, or may be off-centered. The base can be flexible. The loop can be flexible. The loop strip can comprise at least two loops. The at least two loops can be substantially aligned and centered along a length of the first side of the base, or may be off-centered. The base can comprise a perforation guide for indicating where to cut the loop strip. The perforation guide may be a visual or partial perforation guide.

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The Artificial Breast. The artificial breast component of the device comprises at minimum a shell and a filler. The filler is removable from the shell so as to be washable, replaceable and adjustable. When the artificial breast is made of non-synthetic material, it possesses the above-noted advantages to the wearer, and most importantly is highly comfortable for the wearer over extended periods of use.

The Shell. The shell component of the artificial breast is the outer portion that contains the inner portion referenced herein as the filler.

The shell has a distal side that is further from the body of the wearer. The distal side of the shell defines a bra cup, and can be shaped as preferred by the wearer. The distal side of the shell may comprises a felted or woven fabric that maintains a consistent shape and smooth surface texture. For example, the distal side may comprise a felt layer configured in a stable shape to form the bra cup. The felt layer may comprise felted Alpaca fiber, wool fiber, rayon fiber, or a combination thereof. Exemplary felt layer materials comprise protein-based fibers such as Alpaca and wool.

The distal side of the shell may further comprise an inner and an outer cloth layer, covering the felt layer. Thus, the felt layer is disposed between the inner and outer cloth layer. The outer cloth layer covering the felt layer would thus be adjacent the clothing article when in use, whereas the inner cloth layer covering the felt layer would thus be adjacent the filler. An appropriate cloth weave would include a woven fabric having a smooth surface as well as some flexibility, such as a jersey weave material. The inner and/or outer cloth layers may comprises Alpaca fiber, hemp fiber, wool fiber, cotton fiber, bamboo fiber, silk fiber, rayon fiber, or a combination thereof. Exemplary inner and outer cloth materials include plant-based (carbohydrate-based) fibers such as cotton, bamboo, and rayon.

The shell has a proximal side, opposing the distal side. The proximal side which is the side that is most proximal to the wearer, for example adjacent the wearer's skin when in use. The proximal side is flexible, and thus configurable to the conformation and contours of the wearer's body.

The proximal side of the shell of the artificial breast may comprise a flexible layer so as to permit conformation to the wearer. The proximal side of the shell is the side to be placed adjacent to (proximal to) the wearer's torso, and in particular the wearer's skin. Thus the softness of the material is given consideration for the proximal side. The flexible layer of the proximal side of the shell is adequately flexible to permit expansion or accommodation relative to the amount of filler disposed within the shell. This permits the shell to conform to the contours of the wearer's body, even when different types, amounts, weights, or shapes of filler are used.

The flexible layer of the proximal side of the shell defines an opening for inserting and removing filler within the shell. The flexible layer may comprise Alpaca fiber, hemp fiber, wool fiber, cotton fiber, bamboo fiber, silk fiber, rayon fiber, or a combination thereof. An exemplary configuration for the opening is an overlapping flap comprised of jersey weave fabric, optionally wherein the jersey weave fabric comprises cotton, rayon or silk.

Felted Form Shaping. The shell of the artificial breast can be shaped and sized to provide a smooth outer appearance, achieved in certain embodiments by using certain materials and methods to form the felt layer of the distal side of the shell of the artificial breast. For example, a compressed felted form may be shaped, for example by steaming, and may be used such that the artificial breast closely approximates a wearer's other breast. In another example, the compressed felted form may be shaped and used such that the artificial breast is used on either side of the wearer's chest, and may be shaped (e.g., by steaming) so that each breast is closely matched and provides similarity and symmetry of size and shape. The felted form shape (felt layer) forms the basis of the shaped bra cup of the distal side of the shell of the artificial breast. The felt layer is then placed adjacent to a form fitted liner (inner cloth layer) which provides an interface between the felt layer and the filler used to fill the space within the shell. An all-natural material may be used as the outer cloth layer on the exterior of the artificial breast.

The inner cloth layer, felt layer and outer cloth layers may be stitched in place, moulded and/or steamed to maintain form, once secured within the clothing article, such as a bra, bra cup, or inner shelf design clothing item, to suit the wearer's shape and size requirements.

The Filler. The filler is removably disposed within the shell of the artificial breast. The filler may be provided in an amount configured to provide the desired form and firmness to the artificial breast.

In some embodiments, the filler may be a spun fiber that entrains air and hence volume therein. The filler can be fluffed after washing or after compression from multiple uses. Exemplary fibers include Alpaca fiber, hemp fiber, wool fiber, cotton fiber, bamboo fiber, silk fiber, rayon fiber, or a combination thereof. Protein-based fibers such as derived from Alpaca or wool such as lamb's wool, Marino wool or cashmere, provide exemplary light and loose fibers appropriate for the filler.

An exemplary material used for the filler comprises a loose fiber-fill of Alpaca fiber, in amounts sufficient to prevent gaps and follow the contours of the wearer's body.

To permit the wearer extra options and flexibility, set amounts of additional filler may be ready to deploy as part of the device. Additional weights of filler, such as an incremental 1 ounce or 2 ounce option, may be provided at purchase so that if a firmer or larger breast appearance is desired, additional filler may be deployed.

Optional Fill Components. In addition to the filler as described herein, the shell may also be configured to retain other materials therein if a particular size, shape or additional weight is desired. Materials that may act as a weight within the shell of the artificial breast includes porous rock, clay, wood, or charcoal, which permit a higher density than would be accomplished by simply adding additional filler. In such an instance, the filler may be used to swathe or surround the weight or porous rock, clay, wood, charcoal prior to its insertion into the shell. Other materials that may act as a weight within the shell of the artificial breast include granular and particulate materials, such as sand, which may be formed of porous rocks or clays, sawdust, or wood chips. When granular and particulate materials are used, they may be disposed within a pouch, sack, or other flexible container that is sized and shaped to fit within the shell of the artificial breast so that the granular and particulate materials are securely contained within the artificial breast. A wearer of the artificial breast may choose to add additional weight to their artificial breast so that the artificial breast is approximately the same weight as the wearer's other breast; or so that the artificial breast is approximately the same weight as the breast that was removed (wholly, or in part). The materials selected to provide this additional weight may possess any one or combination of anti-bacterial, hypoallergenic, moisture wicking, biodegradable, breathable, and thermal regulating properties.

Non-synthetic Materials. In the shell and filler components of the artificial breast, the use of non-synthetic materials is associated one or more of the following advantages. Non-synthetic material may have anti-bacterial, hypoallergenic, moisture wicking, biodegradable, breathable, or thermal regulating properties, or a combination of any or all of these properties.

The sources of non-synthetic materials noted above include Alpaca, hemp, wool, cotton, bamboo, silk, rayon, or blends and combinations thereof. For some purposes, such as for filler, a spun thread or loose weaver permitting entrained air to produce fluff or volume may be desired. For other purposes, a felted fiber is desired, so as to permit shape and structure. Still further, some purposes require a smooth surface and serve as an outer protective layer of cloth, or an inner interface, in which case a weave, such as a jersey weave, or other appropriate fabric weave or structure may be used.

Notably, threads, ribbons, fasteners and other notions used in the production of the device described herein need not be formed of non-synthetic materials. For these components, other considerations such as strength of thread, availability, cost, and durability are taken into account.

Threads used for attachment of materials may optionally comprise some elastin, such as a fleecy nylon thread. TruLock™ thread may be utilized to advantageously affix expandable and flexible materials or weaves, such as jersey weave cotton. Such thread may permit some stretch in the device material as well as at the seams during manufacture.

Threads and notions (such as ribbons and straps) may comprise polyester blends without having impact on skin-facing materials.

Fastening Straps. The device comprises at least two fastening straps coupled to the artificial breast. There may be two, three, four or more fastening straps present and coupled to the artificial breast, and the more such straps, the more loops may be present for fastening the artificial breast to an article of clothing. The fastening straps may be made of a soft flexible material, such as cotton, rayon, bamboo, silk, etc. The fastening straps may be in the shape of a ribbon or strip. The fastening straps are coupled to, for example permanently attached to, the artificial breast at a first end. At the second end of each strap, opposing the first end, a fastener is disposed.

The configuration of the second end of the fastening strap permits interaction and engagement with a loop so that it may be releasably fastened to the loop.

The Fastener. The second end of the fastening straps comprise a fastener for coupling each fastening strap through its respective loop. The fastener may be, for example, a two part cooperating fastener, with the two parts disposed in sequence at the second end of the fastening strap. The fastener may be of any acceptable type, such as a snap, a hook and loop fastener (for example, Velcro™), or an adhesive fastener.

The Feed Tab. To facilitate easy interconnection and release of the artificial breast to the article of clothing, the second end of the fastening strap may be fitted with a feed tab, terminal to the fastener, to assist the wearer to feed the fastening strap into the loop. The feed tab may be configured with a tapered shape to readily permit threading of the second end of the fastening strap into the loop. The feed tab may be rigid or resilient so that the wearer may readily grasp the tab for the threading of the tab through the loop.

In one embodiment, the feed tab is curved to conform with the contour of the artificial breast when it is in place within the clothing article. Specifically, the feed tab may curve upwardly from the fastening strap to facilitate easy connection and disconnection of the fastener regardless of the dexterity of the wearer. As a further advantage, the curve of such a feed tab follows the curve of the artificial breast and thus is less detectable and less likely to poke or cause discomfort to the wearer when in place.

In an exemplary embodiment, the fastener comprises a snap having a snap seat and a snap cap, and the feed tab is integral to the terminal end of the snap cap. Such a feed tab may be tapered for ease of connection with the loop, and may also be upwardly curved to reflect the curve of the artificial breast when in place in the clothing article. The tapered and the upwardly curved configuration of the terminal end of such a snap cap advantageously permits easy connection and disconnection of the snap cap from the snap seat. In an alternative embodiment, the feed tab may be rounded at its end, and rigid so as to be readily grasped by the wearer for insertion through a loop.

In another embodiment, the feed tab comprises a body having a first end and a second end, where the first end is configured to couple to a fastening strap (for example, configured to be permanently, integrally, or removably attached), and the second end comprises a tapered tab configured to feed the feed tab through a loop. The tapered tab may be curved upwardly from the body, the shape of which can facilitate threading the feed tab through the loop. The body of the feed tab, including the tapered tab, may be rigid or resilient, which can also facilitate threading the feed tab through the loop. The body of the feed tab may be further configured to couple to, or be integral to a first part of a two-part cooperating fastener. In an embodiment, the first end of the body is configured to couple to, or be integral to the first part of the two-part cooperating fastener, where the fastener can releasably fasten the fastening strap through its respective loop. The two-part cooperating fastener may be a snap, a hook and loop fastener, or an adhesive fastener. When the fastener is a snap, the feed tab may be integral to the snap. For example, when the snap has a snap seat and a snap cap, the first end of the body of the feed tab may be integral to the snap seat or snap cap.

Loops. At least two loops are associated with the device, each configured to couple to a clothing article by adhesion (such as with heat activated adhesive interfacing), sewing, or other acceptable means. In some embodiments, each loop is coupled to a base (for example, by adhesion or sewing), where the base is configured to couple to the clothing article (for example, by adhesion, such as with heat activated adhesive interfacing, sewing, or other acceptable means). The at least two loops and/or the base may be made of a soft flexible material, such as cotton, rayon, bamboo, silk, a synthetic material, etc. The base may be in the shape of a shortened ribbon or strip. In some embodiments, each loop is centered, or substantially centered on the base. Two, three, four or more loops may be present to help position one artificial breast, and thus each loop is coupled to the subject article of clothing in an appropriate location to receive the fastening strap. In this way, each loop permits the artificial breast to be secured via the respective fastening strap. By requiring at least two loops, an artificial breast is secured to the article of clothing, at two or more points of contact. By having more than one point of contact, the artificial breast is less prone to unwanted shifting and movement when the wearer goes about daily activities.

A strip of loops (a “loop strip”) may be provided; for example, a strip of aligned loops. The loop strip may comprise a base to which the loops are coupled. As above, the at least two loops and/or the base may be made of a soft flexible material, such as cotton, rayon, bamboo, silk, a synthetic material, etc. The base may be in the shape of a ribbon or strip. In some embodiments, the loops of the loop strip are regularly spaced relative to each other, for example along the length of the base. In some embodiments, the loops of the loop strip are centered, or substantially centered on the base of the strip; for example, along the length or longitudinal axis of the base. Such a loop strip may be secured (for example, via the base) to the interior edge of the clothing article in the side and/or lower regions corresponding to the locations of the respective fastening straps. The loop strip may be secured as-is, or individual loops can be cut from such a strip for individual placement; for example, by cutting the base of the strip between each loop or pair of loops coupled thereto. Whether used in a loop strip, or as individual loops, the loops may be coupled to the interior edge of the clothing article, such as to a bra cup or inner shelf design of any article of clothing.

Interaction of Loops with Fastening Straps. Each of the at least two fastening straps is configured to releasably fasten to each of the at least two loops. Each fastening strap is positioned in a location adjacent to the respective loop when secured to an article of clothing. Depending on the wearer's preference, optional loops may be coupled with the article of clothing, so as to suit more than one differently configured artificial breast having more or fewer fastening straps thereon, or fastening straps in slightly different locations. For example, one type of artificial breast of a certain size, shape or weight may be selected for a certain occasion, whereas on another occasion the same wearer may select another artificial breast of a different size, shape or weight. Athletic endeavors, formal occasions, and casual occasions may be accommodated with different artificial breasts being used. The same clothing article, such as a bra, can accommodate different artificial breasts by being coupled with a variety of loop options for differing fastening strap positions, so that more than one different option for the artificial breast can be selected, and either can achieve secure positioning when in use.

When a feed tab is present on the second end of the fastening strap, the feed tab is threaded through the loop until the fastener is in place. When a two-part co-operating fastener is used, the first of the two parts is threaded through the loop, and then connected to the remaining part of the fastener. All fastening strap and loop pairings are then connected accordingly, so that the artificial breast is held securely in place. To remove the artificial breast from the clothing article to which the loops are attached, the opposite actions are taken in that the cooperating fastener parts are detached, and pulled back through the loop to separate the artificial breast from the clothing article.

An exemplary fastening system comprises a two part system in which a strip contains multiple loops (a loop strip), comprising flat loops about 1-2 cm long (such as about 1.3 cm), spaced at a distance of 1-2 cm apart (such as about 1.3 cm), and substantially centered along the longitudinal axis of the strip, wherein each section of the loop strip can be used independently or in multiple loop units, as desired. The loop strip may be configured or designed to accept a fastening strap having a snap fastener and a terminal feed tab. The fastening strap may be formed of 1-2 cm (for example, about 1.6 cm) ribbon with a finished slightly rigid, curved, terminal feed tab end followed by a male snap head (or snap “seat”), a 1-2 cm space (for example about 1.3 cm) and then the female snap head (or snap “cap”). An exemplary overall measurement of the fastening strap is from 4-8 cm in length, such as about 7.5 cm.

The Clothing Article. The clothing article to which the loops are configured to couple may be any article to be worn in the area adjacent to the upper torso and in particular the breast area. Such articles of clothing include but are not limited to a top such as a shirt or sweater, a bra such as a sports bra or support bra, a dress for either formal or casual purposes, an item of lingerie such as a slip or camisole, a bralette, or a swimsuit of either a one-piece configuration, or a bikini or two-piece configuration. When used in a swimsuit, the filler removably disposed within the shell of the artificial breast may be comprised of adaptable materials suitable for use in water, such that the artificial breast may maintain its size, shape, and/or form.

The clothing article may be one which comprises an inner shelf comprising a seam or other shelf-type configuration above which the breast area resides. When the clothing article has an upper shelf, coupling the loops to this upper shelf region is particularly advantageous because of the way in which the coupling may be accomplished in a less detectable manner due to the existence of a seam or other fabric configuration. In such instances, the loops can be coupled to the clothing article along the inner shelf.

Methods of Securing the Device. Methods are described herein that permit a wearer to secure the described device to an article of clothing, and in particular an article of clothing comprising an inner shelf region. Briefly, the at least two loops are coupled to the inner shelf of the clothing article, for example by sewing to the inner shelf a ribbon containing loops thereon, or by affixing individual loops along a seam defining the inner shelf region.

The device is then able to be fastened at each of the at least two fastening straps to the respective loops to hold the device securely in the appropriate position. Thus, the artificial breast can remain affixed to the inner shelf of the clothing article so that the wearer may move freely without concern for unexpected movement or release of the artificial breast.

A Kit of Parts. The device may be provided to consumers as a kit of parts. This may be particularly advantageous for permitting customization and for home assembly. Such a kit may comprise an artificial breast comprising a shell and a filler that is removably disposed within the shell. The kit can be provided with the at least two fastening straps already coupled to the artificial breast, or configured to be attached by the consumer, such as for being sewn onto the artificial breast by the consumer once the desired locations of the fastening straps are determined in consideration of the subject clothing article.

The kit would include the at least two loops, which are able to couple to a clothing article, such as by adhesion or sewing. By providing the loops in the kit for the consumer to attach, the consumer may advantageously assess optimal placement within the comfort and privacy of their own home. Further, the kit may be supplied with extra loops which may be installed in multiple clothing articles, so that the same artificial breast can be used in conjunction with multiple different articles of clothing. Such a kit can be provided with a user manual including instructions for installation and use, such as for laundering, drying, etc.

Clothing Articles with Pre-Affixed Devices. It is possible that the device may come pre-affixed by a manufacturer or by custom installation to a clothing article. For example, a bra or bathing suit can be manufactured with the device pre-affixed thereto, but nevertheless still removable for purposes of easy laundering. Such a bra may conveniently be a front-closure bra, which readily permits correct placement of the device adjacent to the wearer's skin regardless of whether the wearer faces dexterity challenges, for example attributable to surgery, age, or other reasons. Front closure advantageously negates the wearer having to attach back bra clasps together at the front followed by a rotation of the clasped bra 180 degrees around the torso into the wearing position. Such rotation of a bra can be irritating to individuals following recovery from surgery.

The Fastening System, Feed Tab, Loop Strip, and Other Articles. The fastening system described herein, comprising a fastening strap with fastener configured to couple to a first article and a loop configured to be coupled to a second article such that the fastening strap releasably fastens to the loop to secure the first article to the second article, may be used to secure together articles other than clothing. The fastening system may be used to secure a first article, such as a couch cushion to a second article, such as a couch or cushion cover; or to secure a duvet cover to a duvet. For example, the fastening strap with fastener may be coupled to the couch cushion or the duvet (for example, by adhesion, such as with heat activated adhesive interfacing, sewing, or other acceptable means), and the loop may be coupled to the couch or cushion cover, or to the duvet cover (for example, by adhesion, such as with heat activated adhesive interfacing, sewing, or other acceptable means). As such, the cushion may secured to the couch, the cushion cover may be held in place around the cushion, or the duvet cover may be held in place around the duvet. Such articles may be manufactured with the fastening system pre-affixed thereto, or the fastening system may be sold separately for consumers to affix the fastening system to previously purchased articles.

The feed tab described herein, comprising a body having a first end and a second end where the first end is configured to couple to a fastening strap for coupling to a first article and the second end comprises a tapered tab configured to feed the feed tab through a loop for coupling to a second article, may be used to secure together clothing articles and the prosthetic device described herein; and/or may be used to secure together articles other than clothing. The loop strip described herein, comprising a loop coupled to a base where the loop is configured to receive a fastening strap coupled to a first article and where the base has a first side to which the loop is coupled and a second side configured to couple to a second article, may also be used to secure together clothing articles and the prosthetic device described herein; and/or may be used to secure together articles other than clothing.

The clothing articles may be any article to be worn in the area adjacent to an upper torso, and in particular the breast area. The clothing article may be one which comprises an inner shelf comprising a seam or other shelf-type configuration above which the breast area resides. Such articles of clothing include but are not limited to a top such as a shirt or sweater, a bra such as a sports bra or support bra, a dress for either formal or casual purposes, an item of lingerie such as a slip or camisole, a bralette, or a swimsuit of either a one-piece configuration, or a bikini or two-piece configuration. The other, first and second articles may include, but are not limited to a couch cushion and couch, a cushion cover and cushion, a duvet cover to a duvet, etc.

Advantages. By way of comparison with certain known prostheses, the present device may possess advantages.

Previously known prostheses do not consistently permit attachments beyond insertion into a bra pocket. Individuals wishing to use a prosthesis device in an existing (unmodified) bra would need to alter such a bra to include a pocket in which a prosthesis may be held. However, because previously known prostheses have no way to couple or affix within the pocket of such an altered bra, the prosthesis would have considerable movement within a bra pocket. When a previously known prosthesis moves around within a bra pocket, it may become misshapen or out of place, causing the wearer considerable irritation. The device described herein does not require a pocket, and is secured at two or more points to avoid excessive movement relative to a bra or other clothing article.

Previously known devices may cause a wearer to become sweaty due to the nature of the silicone and/or polyester materials used. Advantageously, the use of non-synthetic materials in the artificial breast described herein promotes breathability and thermal regulation to minimize sweating when the device is in place adjacent the wearer's body.

Previously known devices may leave gaps between the prosthesis and the wearer's body. Advantageously, embodiments described herein permit the proximal side of the artificial breast to conform to the wearer's body and the option of filler adjustments permit more or less volume to be contained within the shell. The material used for the filler may be, for example, an all-natural loose fiber-fill of Alpaca fiber, which can be used in an amount that prevents the gaps that previously known solid prosthesis may leave. Advantageously, the wearer can control the firmness of the contour by the amount of filler used prosthesis. The wearer may add or reduce the amount of fill to conform with the negative space at varying times during the post-surgical healing process. For example, immediately following surgery when the wearer is tender, a different amount of fill may be uses as compared to once healing is complete, and the wearer is gaining freedom of movement.

Advantageously, the nature of the materials used in the device described herein have a more natural feel when placed next to the body. Further, individuals who have environmental concerns about the use of synthetic materials will feel a sense of relief in the knowledge that the materials used in the artificial breast are non-synthetic.

The fastening system described herein, whether inherent to the device or taken on its own, permits transformation of any article of clothing, such as articles having a bra cup or inner shelf design, into a wearable fashion providing comfort and confidence to the wearer. The wearer advantageously feels confident that the artificial breast is kept securely in place, and in the correct position. Further, the wearer can be confident that the artificial breast will maintain a consistent appearance when installed into different clothing articles by consistent loop and fastening strap placement.

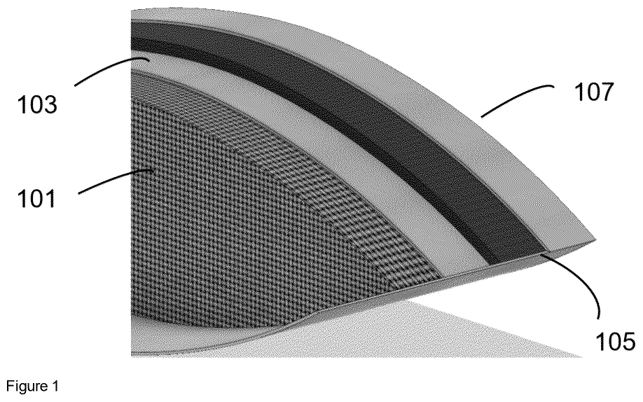

FIG. 1 schematically depicts, in section, the layers within an embodiment of an artificial breast as described herein, including (from left to right) the filler (101) shown as a textured/hatched layer, the inner cloth layer (103) shown in light grey shading, the felt layer (105) shown in black, and the outer cloth layer (107) also shown in light grey shading.

To gain a better understanding of the invention described herein, the following examples are set forth. It should be understood that these examples are for illustrative purposes only. Therefore, they should not limit the scope of this invention in anyway.

### EXAMPLES

### Example 1

### Assembly and Installation of the Prosthetic Device

A method is described for assembly of the prosthetic device, and in particular for preparing the artificial breast from its constituent fabrics and materials. The shape of the artificial breast is determined and maintained by the felt layer of the shell, while the space within the shell is occupied by the filler. It was found that the support provided by the felt layer was maintained over multiple uses, and was retained after laundering. The filler was found to create natural puff needed to fill the negative space within the shell to the desired fullness.

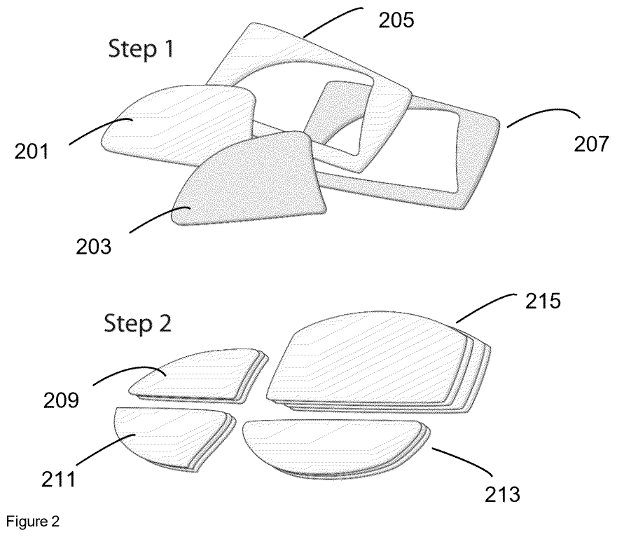

The following steps were conducted, as depicted in FIG. 2 to FIG. 4.

Step 1—Cut out materials from appropriately selected fabrics. Note that seams for darts are sewn with overlock machine so as to smooth out the darts and leave a flat surface. In this way, a flat surface is achieved without evidence of a seam or excess fabric bulk. FIG. 2 illustrates step 1 in the upper panel, showing cutting of similar shaped components (201, 203) of the shell, from different materials (205, 207) that form the shell.

Step 2—Arrange distal side layers of the shell (outer cloth layer, felt layer, inner cloth layer). FIG. 2 illustrates step 2, in which stacks of three different shapes (209, 211, 213) of components of the distal side layers cut to the same shape, are arranged in alignment for sewing. The felt layer comprises alpaca felt. The felt layer is sandwiched between 2 cotton or bamboo fabrics (the outer cloth layer and the inner cloth layer). The upper left stack (209) and two lower stacks (211, 213) of layers are arranged to have three pieces each represent the distal side layers.

The proximal side pieces are shown in the stack (215) of cut pieces as the upper right stack in Step 2 of FIG. 2. These proximal side pieces form the proximal side of the shell having a right side and a left side with overlap. The proximal side pieces are cut about 1-2 cm smaller in width and double length to create overlap of both pieces when folded, therefore not requiring a closure to maintain the filler within the shell. The proximal side pieces are cut so that they can be folded to a double thickness, thereby creating a seamless edge at the opening. The seamless edge is advantageously comfortable and smooth against the skin of the wearer. The cut pieces are assembled to create the shell having a breast shape attributable to the shaping of the felt layer. Access to the interior of the shell for filling with the filler is to be done from the proximal side at the overlapped pieces.

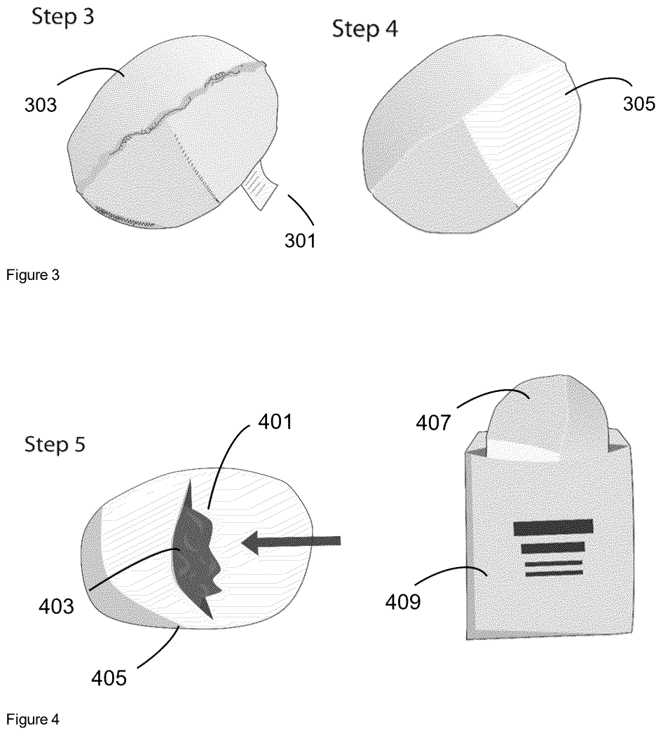

Step 3—The cut pieces are stacked together in alignment for sewing, optionally together with the first end of each fastening strap or a marked opening where the first end of each fastening strap is to be inserted. Optionally, a care instructions/materials tag (301) is attached. The pieces are then sewn, in order to create the shell. The fastening straps (or openings therefor) align to the inside for sewing, so as to be external to the shell once inverted. Interlock stitching is used on a machine fitted with a walking foot, so as to keep the multiple layers of materials aligned so they don't slip. All distal side pieces are sewn together, following which the proximal and distal edges are aligned with the two fastening straps (or openings therefor) and a seam is sewn around the edge (303) of the layers to affix the proximal side to the distal side. The portions comprising the proximal side are overlapping thereby to create a central opening to access the interior of the shell. FIG. 3 shows step 3 from the distal side after sewing and prior to inversion. For a prosthesis in which fastening straps tabs are not included but spaces are left for later affixation, the tabs are added later the appropriate location. In configurations where no fasteners are needed, there is no need to accommodate fastening tabs or straps for later affixation, as they are not required.

The overlock stitching is used to affix the 3 pieces (1 top and 2 bottom) which form the distal side of the shell, which together emulate the shape of a bra cup. A woolly nylon thread may be used to give the seams stretch. The overlock stitch, makes it easier to smooth the shell's distal side shape.

Using darts, when attaching the pieces that form the proximal side of the shell, gives good definition and structure. Identifying the depth of the breast (cup size) by the size of the dart gives an appropriate shape and “arch curve” to the cup.

Step 4—Turn finished piece right-side out (inversion) via the overlap opening on the proximal side of the shell. Smooth out the seam, steam to “set” the alpaca felt shape before filling. Further, steaming also serves to sanitize the product so formed. FIG. 3 shows step 4 in which the inverted (right-side out) shell (305) is shown from the distal side.

Step 5—Add filler in 1-2 oz. increments (approximately 30-60 g). FIG. 4 (left side) illustrates how filler (401) is added through the overlapping sides (403) defining an opening on the proximal side of the shell (405). For distribution and/or delivery, the artificial breast (407) can be packaged in a paper sleeve (409) with stamped markings, showing design and size, as depicted in FIG. 4 (right side). In the depicted embodiment, there is no need for straps or fasteners, which are optional for the artificial breast.

Once manufactured, the device may be installed within a clothing article.

### Example 2

### Fastening System for Securing Prosthetic Device in a Clothing Article

A method is described for installation of an artificial breast in an article of clothing such as in a bra.

FIG. 5 depicts a ribbon (501) having multiple loops (503) substantially centered and disposed along the length thereof, also referred to herein as a “loop strip”, for placement within a clothing article to which the fastening straps are fastened to secure the artificial breast. The loop strip may be provided with visual or partial perforations between each loop or pair of loops, to guide a user to cut in a pre-designated location that would not disrupt the integrity of a loop when cut. A user may wish to couple an entire loop strip along a bra cup area of a clothing article such as a bra, or along a shelf-type seam in a clothing article such as a top.

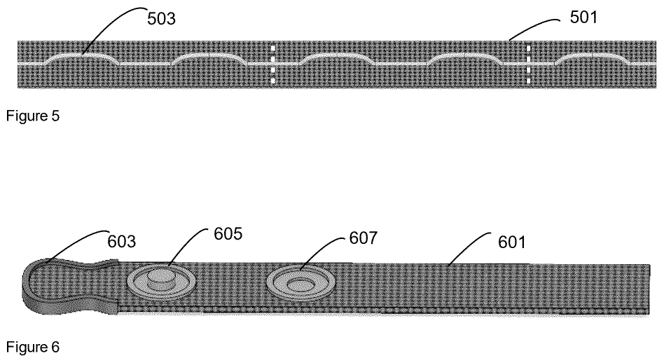

FIG. 6 depicts an embodiment of the fastening strap (601) with a rigid and rounded feed tab (603) and wherein the fastener is a snap having two parts, a seat (605) optionally referenced as a “male” component, and a cap (607) optionally referenced as a “female” component.

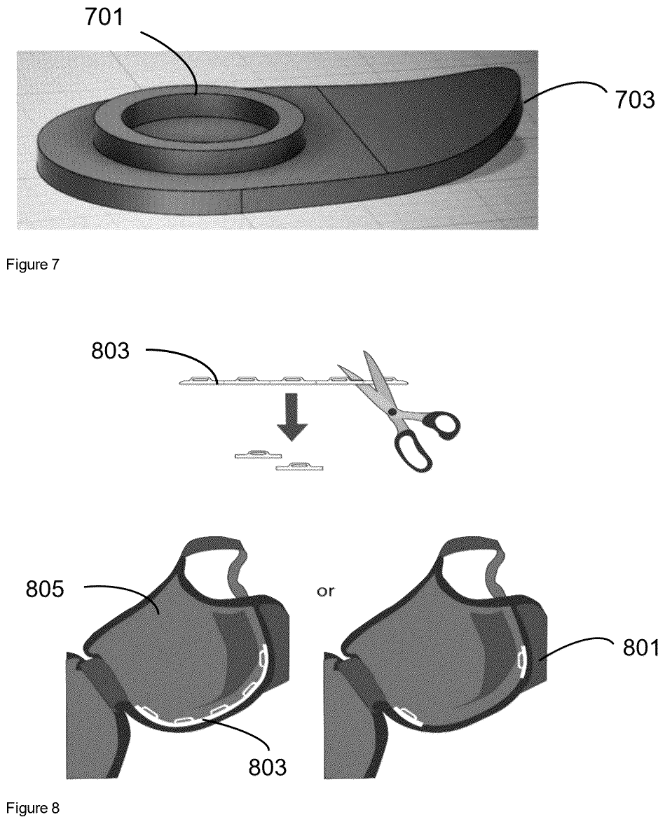

FIG. 7 depicts an embodiment of one part of a two part snap for use as a fastener on a fastening strap. The snap component (701) is depicted as having an integral tapered and curved feed tab (703). The snap component with the integral feed tab may comprise either the seat or the cap component of the snap. It is located at the terminus of the second end of a fastening strap, so that the curved feed tab is disposed to direct the fastening strap through a loop.

FIG. 8 shows options for the placement of individual loops (801) or a loop strip (803) comprising multiple loops in a bra (805). The loops can be affixed to the interior of a bra, at the lower and/or side perimeter of the cup. Coupling a loop strip (803) or individual loops (801) to the body-facing (interior) side of the clothing article can be accomplished using sewing, adhesive, heat tape, or other means that renders loops securely in place on the clothing article.

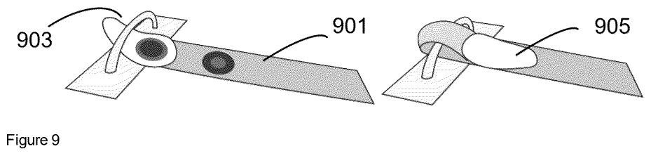

The fastening system in this example secures the artificial breast to the clothing article in a reversible manner. The loop strip (803) is comprised of flat loops of about ½″ long spaced at a distance of ½″ apart. Each loop section of the loop strip can be used independently by cutting the loops (801) apart, or the intact loop strip (803) may be used in multiple units, as desired. The loop strip depicted in this example is designed to accept a fastening strap as depicted in FIG. 9.

FIG. 9 depicts a fastening strap during and after fastening to a loop. A fastening strap (901) is shown which is constructed of a ⅝″ ribbon with a finished slightly rigid, curved, feed tab (903) end followed by or integral to a male snap head, followed by a ½″ space and then the female snap head. Overall measurement of the depicted fastening strap (901) is about 3″ in length. FIG. 9 (right) depicts the two parts of the snap (905) once connected together after the fastening strap is fed through a loop.

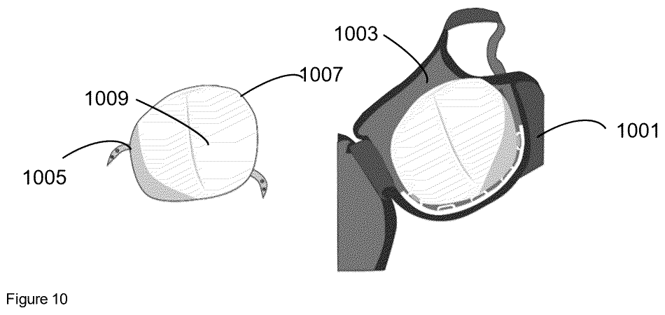

FIG. 10 depicts an embodiment of the artificial breast before and after fastening to the loops in a clothing article. A loop strip (1001) is secured to the interior edge of the bra (1003) or other article of clothing to accept the fastening strap (1005) which is attached to outer shell of the artificial breast (1007). The feed tab end of the fastening strap is threaded through a loop, as facilitated by the slightly rigid, curved and tapered end of the tab. The two parts of the snap are then fastened. The curved end of the tapered feed tab conveniently follows the curve of the artificial breast, and thus does not stick out once the snap is fastened. The fastening of the snap creates a secure but easily reversible connection between the artificial breast (1007) and the bra (1003) or article of clothing. The proximal side of the shell is shown with the overlap opening (1009) depicted, through which filler is added and removed.

The fastening system described in this example may be used to keep the presently described artificial breast in place, but may also be used for other prostheses to which the fastening straps may be coupled. Thus, other breast prostheses may be secured in place in this way at an appropriate position within a bra cup or inner shelf region of an article of clothing, thereby improving comfort and performance.

### Example 3

### Loop Strip Configurations

The loop strip can be used as a component of the with the fastening system for installation of an artificial breast in an article of clothing such as in a bra. A variety of loop strip configurations are possible.

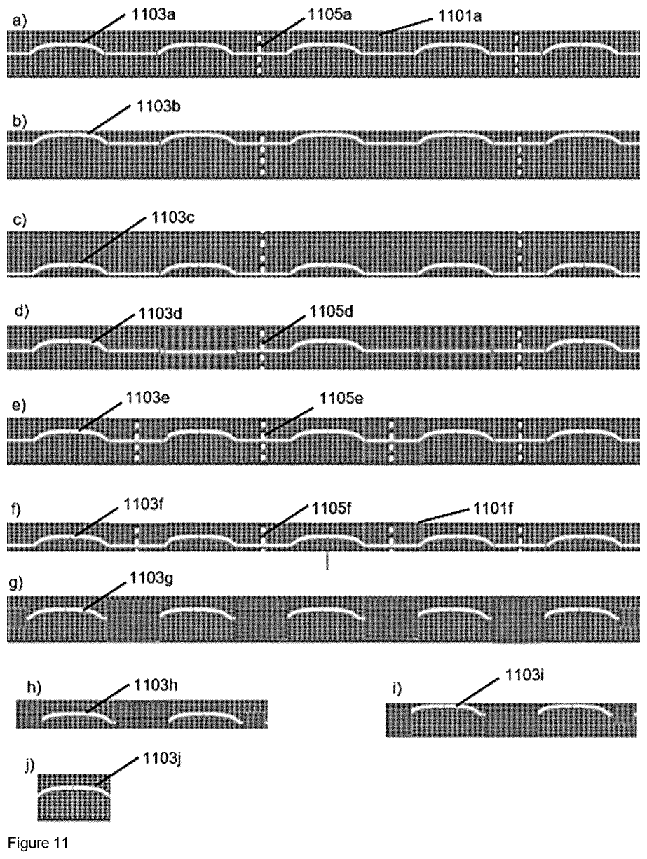

FIG. 11 depicts stylized loop strip configurations (a) to (j) having one or more loop disposed on a base ribbon. The depicted loop strip configurations are not limiting, and it is emphasized that bases and loops may be of a wide variety of widths and proportions beyond those depicted here, provided the loop strip is suitable for placement within a clothing article.

Loop strip configuration (a) depicts a section of a continuous ribbon (1101a) having multiple loops (1103a) substantially centered and disposed along its length thereof, with a regularly spaced visual or partial perforation guide (1105a) indicating where a user sewing the loop strip onto the article of clothing may or may not wish to cut the loop strip. The perforation guide may be indicated visually such as by a different colour incorporated in the ribbon fabric, or may be indicated structurally, such as by partial perforation of the ribbon fabric.

Configuration (b) depicts a continuous loop strip having multiple loops (1103b) in an off-center placement. Configuration (c) depicts a continuous loop strip having multiple loops (1103c) in an edge placement. Configuration (d) depicts a continuous loop strip in which loops (1103d) have a central placement, but with greater distance between loops, and only one loop between perforation guides (1105d). Configuration (e) depicts a continuous loop strip in which loops (1103e) have a central placement, with periodicity similar to that of configuration (a) but perforation guides (1105e) between each loop. Configuration (f) depicts a continuous loop strip in which loops (1103f) have an off-center placement, with perforation guide (1105f) periodicity similar to that of configuration (a) but with a narrower ribbon (1101f). Configuration (g) depicts a loop strip with loops (1103g) having central placement but without any perforation guide or loop material between loops. Configuration (h) depicts a non-continuous loop strip having two loops (1103h) with off-center placement. Configuration (i) depicts a non-continuous loop strip having two loops (1103i) with edge placement. Configuration (j) depicts a non-continuous loop strip having a single loop (1103j) with central placement. Further configurations are possible, beyond those depicted here.

When used as a component of a fastening system within an article of clothing, such as a bra, a continuous loop strip comprising multiple loops coupled to a base may be placed along a bra cup edge, or discrete non-continuous loop strips comprising at least one loop coupled to a base may be placed periodically along a bra cup edge where needed.

### Example 4

### Configuration with Compression Fit

According to certain configurations of the artificial breast described herein, there is no requirement for fasteners. The artificial breast is configured to suit the needs of the wearer, and it is the filler placement within the shell that provides compression fit to the user, for example when placed within the curved cup of an existing clothing article such as a bra, bralette, or bathing suit. Such an article may be formed with curvature by forming the distal side of the shell as a horizontally approximately oblong shape comprising an upper portion configured as a semi-oblong, and two lower oblong quadrants forming the lower portion of the oblong shape with an approximately central vertical seam between the quadrants to define the contours of the bra cup.

FIG. 12 is a photograph depicts an inverted version of the shell of such an artificial breast, which is in an approximately oblong shape. The inverted shell is being held up by the inventor in this photograph. This is parallel to the configuration depicted in FIG. 4, for which there is no requirement for any a fastener(s) nor for a fastening system. The upper semi-oblong is sewn together with a horizontal seam to the lower semi-oblong that is formed from two oblong quadrants (sewn together with a vertical seam) which is shaped to approximate the size and configuration of the wearer. The interior of the shell is depicted here (turned in-side out), such that the seams will face inwardly to receive the filler, and thus will not show underneath the article of clothing worn by the wearer. The felted Alpaca fabric used to form the interior of the shell can be seen.

FIG. 13 is a photograph depicting the non-inverted version (turned right-side out) of the artificial breast as shown in FIGS. 4 and 12. The artificial breast contains the filler therein. The outer fabric is a cotton/bamboo, and the seams are turned inward and thus are minimized on the finished article. The upper semi-oblong portion can be made to different heights as one way to determine size and configuration of the resulting artificial breast. In this way the oblong need not be symmetrical along its horizontal axis, as the upper semi-oblong portion may be taller or shorter than the semi-oblong that is formed by the two lower oblong quadrants when sewn together.

The artificial breast may be stuffed with a small, medium, large or very large amount of Alpaca fiber, which is prepared as a loose or spun fiber that may resemble a large cotton ball. The flexibility of the Alpaca fiber allows the fiber to be readily stuffed into the opening on the proximal side of the shell, while maintaining a consolidated shape when contained within the shell. The loose and flexible filler, when contained within the shell, will take on a firm and consolidated shape to fill the space provided within the shell, fully filling out the shell in a way that looks indistinguishable from a breast when worn.

The flexibility of the filler also permits easy removal for airing out or washing, or for substitution with a firmer or less firm filler, as desired. Further, if a porous rock is desired to include, depending on the needs of the user, the loose fiber filler may be wrapped around the rock in a manner that fully encompasses it within the filler prior to insertion into the shell. In this way, a user will be confident that the contents of the shell will stay intact and cause appropriate compression when worn so as to stay in place, without the need for fasteners.

When Alpaca fiber, such as a loose/spun fiber is used as the filler within a felted Alpaca shell, the friction between the two natural fibers on the inside of the artificial breast will work together to maintain the filler intact within the shell.

Among the various aspects described herein are the following.

Aspect 1. An artificial breast comprising a shell and a filler removably disposed within the shell, said artificial breast comprising non-synthetic material; said artificial breast being shaped and configured to fit securely within a clothing article by compression between a wearer and the clothing article to inhibit movement of the artificial breast relative to the clothing article; wherein: the shell comprises a distal side defining a curved bra cup and a proximal side configured to conform to contours of a body and configured to expand, the distal side of the shell comprising an inner felt layer configured to form the bra cup and an outer cloth layer for facing the clothing article when worn, and the filler is removably disposed within the shell in an amount configured to provide form and firmness to the artificial breast and in an amount adequate to cause compression of the artificial breast when worn; wherein the proximal side of the shell defines a flexible opening for disposing the filler within the shell, and said shell flexibly accommodates different amounts of filler.

Aspect 2. The artificial breast of Aspect 1, wherein the non-synthetic material has anti-bacterial, hypoallergenic, moisture wicking, biodegradable, breathable, or thermal regulating properties, or a combination thereof, wherein: the felt layer of the shell comprises felted Alpaca fiber, wool fiber, rayon fiber, or a combination thereof; and the filler comprises Alpaca fiber, hemp fiber, wool fiber, cotton fiber, bamboo fiber, silk fiber, rayon fiber, or a combination thereof.

Aspect 3. The artificial breast of Aspect 1, wherein the shell further comprises an inner cloth layer comprises Alpaca fiber, hemp fiber, wool fiber, cotton fiber, bamboo fiber, silk fiber, rayon fiber, or a combination thereof.

Aspect 4. The artificial breast of Aspect 1, wherein the proximal side of the shell is configured to expand relative to the amount of filler disposed within the shell to conform the proximal side of the shell to the contours of a body.

Aspect 5. The artificial breast of Aspect 1, wherein the filler comprises Alpaca fiber.

Aspect 6. The artificial breast of Aspect 1, wherein the inner felt layer of the shell comprises felted Alpaca fiber.

Aspect 7. The artificial breast of Aspect 1, wherein the proximal side of the shell comprises bamboo and/or cotton.

Aspect 8. The artificial breast of Aspect 1, wherein the distal side of the shell is formed as a horizontally approximately oblong shape comprising an upper portion configured as a semi-oblong, and two lower oblong quadrants forming the lower portion of the oblong shape with an approximately central vertical seam between the quadrants to define the contours of the bra cup.

Aspect 9. The artificial breast of Aspect 9, wherein the upper portion of the oblong shape of the distal side of the shell is configured to the wearer's desired shape and volume of bra cup.

Aspect 10. The artificial breast of Aspect 8, wherein the upper portion and the two lower portions of the oblong shape of the distal side of the shell are each configured to the wearer's desired shape and volume of bra cup.

Aspect 11. The artificial breast of Aspect 1, wherein the bra cup size and shape is suited to the wearer who has had a full mastectomy, a partial mastectomy, and/or who wishes to supplement existing breast volume.

Aspect 12. The artificial breast of Aspect 1, wherein the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn.

Aspect 13. The artificial breast of Aspect 8, wherein the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn.

Aspect 14. The artificial breast of Aspect 1, further comprises a weight disposed within the shell.

Aspect 15. The artificial breast of Aspect 14, wherein the weight is a porous rock.

Aspect 16. The artificial breast of Aspect 1, wherein the clothing article within which the artificial breast is to be worn comprises a top, a bra, a top, a dress, lingerie, a bralette, or a swimsuit.

Aspect 17. The artificial breast of Aspect 1, further comprising at least two fastening straps for securing the artificial breast within a clothing article having loops configured to receive said fastening straps, wherein said fastening straps comprise a ribbon having a first end and a second end, the first end coupling to the shell of the artificial breast; and the second end comprising a feed tab and a fastener, the feed tab being configured for feeding the second end of each of the fastening straps through the respective loop of the clothing article.

Aspect 18. The artificial breast of Aspect 17, wherein the fastener is a snap, a hook and loop fastener, or an adhesive fastener.

Aspect 19. The artificial breast of Aspect 17, wherein the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn.

Aspect 20. The artificial breast of Aspect 17, wherein the distal side of the shell is formed as a horizontally approximately oblong shape comprising an upper portion configured as a semi-oblong, and two lower oblong quadrants forming the lower portion of the oblong shape with an approximately central vertical seam between the quadrants to define the contours of the bra cup.

Aspect 21. A prosthetic device, comprising: an artificial breast comprising a shell and a filler removably disposed within the shell, said artificial breast comprising non-synthetic material; at least two fastening straps coupled to the artificial breast; and at least two loops configured to couple to a clothing article; wherein each of the at least two fastening straps is configured to releasably fasten to each of the at least two loops, respectively, to secure the artificial breast to the clothing article and inhibit movement of the artificial breast relative to the clothing article.

Aspect 22. The device of Aspect 21, wherein the non-synthetic material has anti-bacterial, hypoallergenic, moisture wicking, biodegradable, breathable, or thermal regulating properties, or a combination thereof.

Aspect 23. The device of Aspect 21 or 22, wherein: the shell comprises a distal side defining a bra cup and a proximal side configured to conform to contours of a body; and the filler is removably disposed within the shell in an amount configured to provide form and firmness to the artificial breast.

Aspect 24. The device of Aspect 23, wherein the filler comprises Alpaca fiber, hemp fiber, wool fiber, cotton fiber, bamboo fiber, silk fiber, rayon fiber, or a combination thereof.

Aspect 25. The device of Aspect 23 or 24, wherein the distal side of the shell comprises a felt layer, the felt layer being configured to form the bra cup.

Aspect 26. The device of Aspect 25, wherein the felt layer comprises Alpaca fiber, wool fiber, rayon fiber, or a combination thereof.

Aspect 27. The device of any one of Aspects 23 to 26, wherein the distal side of the shell further comprises an inner and an outer cloth layer, the felt layer being disposed therebetween.

Aspect 28. The device of Aspect 27, wherein the inner cloth layer comprises Alpaca fiber, hemp fiber, wool fiber, cotton fiber, bamboo fiber, silk fiber, rayon fiber, or a combination thereof.

Aspect 29. The device of any one of Aspects 23 to 28, wherein the proximal side of the shell comprises a flexible layer, the flexible layer being configured to expand relative to the amount of filler disposed within the shell to conform the proximal side of the shell to the contours of a body.

Aspect 30. The device of Aspect 29, wherein the flexible layer defines an opening for disposing the filler within the shell.

Aspect 31. The device of Aspect 29 or 30, wherein the flexible layer comprises Alpaca fiber, hemp fiber, wool fiber, cotton fiber, bamboo fiber, silk fiber, rayon fiber, or a combination thereof.

Aspect 32. The device of any one of Aspects 21 to 31, wherein each of the at least two fastening straps comprises: a ribbon having a first end and a second end, the first end coupling to the artificial breast; and the second end comprising a feed tab, the feed tab being configured for feeding the second end of each of the fastening straps through the respective loop.

Aspect 33. The device of Aspect 32, wherein the feed tab is rigid or resilient.

Aspect 34. The device of Aspect 32 or 33, wherein the feed tab is upwardly curved relative to the fastening strap to conform with the contour of the artificial breast.

Aspect 35. The device of any one of Aspects 32 to 34, wherein the second end of each of the at least two fastening straps further comprises a fastener for coupling each of the at least two fastening straps through the respective loop.

Aspect 36. The device of Aspect 35, wherein the fastener is a snap, a hook and loop fastener, or an adhesive fastener.

Aspect 37. The device of any one of Aspects 21 to 36, wherein the at least two loops comprise: each of the at least two loops coupled to a base, the base having a first side to which the loop is coupled and a second side configured to couple to the clothing article.

Aspect 38. The device of Aspect 37, wherein the loop is substantially centered on the base.

Aspect 39. The device of any one of Aspects 21 to 36, wherein the at least two loops comprise: a loop strip comprising the at least two loops and a base, the base having a first side to which the at least two loops are coupled and a second side configured to couple to the clothing article.

Aspect 40. The device of Aspect 39, wherein the at least two loops are substantially aligned and centered along a length of the first side of the base.

Aspect 41. The device of Aspect 39 or 40, wherein the base comprises a perforation guide for indicating where to cut the loop strip.

Aspect 42. The device of any one of Aspects 37 to 41, wherein the base is flexible.

Aspect 43. The device of any one of Aspects 21 to 42, wherein the artificial breast further comprises a weight disposed within the shell.

Aspect 44. The device of Aspect 43, wherein the weight is a porous rock.

Aspect 45. The device of any one of Aspects 21 to 44, comprising at least three fastening straps coupled to the artificial breast, and at least three loops configured to couple to the clothing article.

Aspect 46. A fastening system comprising: a fastening strap configured to couple to a first article, the fastening strap comprising a fastener disposed thereon; and a loop configured to be coupled to a second article; the fastening strap being configured to releasably fasten to the loop to secure the first article to the second article.

Aspect 47. The system of Aspect 26, wherein the fastening strap comprises a flexible ribbon, said fastening strap having a first end and a second end, wherein: the first end is configured for coupling to the first article; and the second end comprises a feed tab, the feed tab being configured to feed the second end of the fastening strap through the loop.

Aspect 48. The system of Aspect 47, wherein the feed tab is rigid or resilient.

Aspect 49. The system of Aspect 47 or 48, wherein the feed tab is curved upwardly from the fastening strap.

Aspect 50. The system of any one of Aspects 46 to 49, wherein the fastener is disposed at the second end of the fastening strap.

Aspect 51. The system of Aspect 50, wherein the fastener is a two part cooperating fastener, with the two parts disposed in sequence at the second end of the fastening strap.

Aspect 52. The system of any one of Aspects 46 to 51, wherein the fastener is a snap, a hook and loop fastener, or an adhesive fastener.

Aspect 53. The system of any one of Aspects 47 to 49, wherein the fastener is a snap and the feed tab is integral to the snap.

Aspect 54. The system of any one of Aspects 46 to 53, wherein the loop comprises: the loop coupled to a base, the base having a first side to which the loop is coupled and a second side configured to couple to the second article.

Aspect 55. The system of Aspect 54, wherein the loop is substantially centered on the base.

Aspect 56. The system of any one of Aspects 46 to 53, wherein the loop comprises: a loop strip comprising at least two loops and a base, the base having a first side to which the at least two loops are coupled and a second side configured to couple to the second article.

Aspect 57. The system of Aspect 56, wherein the at least two loops are substantially aligned and centered along a length of the first side of the base.

Aspect 58. The system of Aspect 56 or 57, wherein the base comprises a perforation guide for indicating where to cut the loop strip.

Aspect 59. The system of any one of Aspects 54 to 58, wherein the base is flexible.

Aspect 60. A method of securing the prosthetic device of any one of Aspects 21 to 45 to a clothing article comprising an inner shelf, the method comprising: coupling the at least two loops to the inner shelf of the clothing article; and fastening the at least two fastening straps to the respective loops, thereby securing the artificial breast to the inner shelf of the clothing article.

Aspect 61. A kit, comprising: an artificial breast comprising a shell and a filler removably disposed within the shell, said artificial breast comprising non-synthetic material; at least two fastening straps configured to couple to the artificial breast; at least two loops configured to couple to a clothing article; each of the at least two fastening straps being configured to releasably fasten to each of the at least two loops, respectively, to secure the artificial breast to the clothing article and inhibit movement of the artificial breast relative to the clothing article; and instructions for installation and use.

Aspect 62. A clothing article comprising the prosthetic device of any one of Aspects 21 to 45, wherein the artificial breast is releasably fastenable to the clothing article.

Aspect 63. The clothing article of Aspect 62, wherein the clothing article comprises an inner shelf and the loops are coupled to the clothing article along the inner shelf.

Aspect 64. The clothing article of Aspect 62 or 63, wherein the clothing article is a top, a bra, a dress, lingerie, a bralette, or a swimsuit.

Aspect 65. A bra comprising the prosthetic device of any one of Aspects 21 to 45.

Aspect 66. The bra of Aspect 65, comprising a front closure.

Aspect 67. Use of the prosthetic device of any one of Aspects 21 to 45 in a clothing article.

Aspect 68. The use of Aspect 67, wherein the clothing article is a top, a bra, a dress, a lingerie article, a bralette, or a swimsuit.

Aspect 69. The use of Aspect 67 or 68, wherein the clothing article comprises an inner shelf.

Aspect 70. The use of Aspect 68 or 69, wherein the clothing article is a bra.

Aspect 71. A feed tab, comprising: a body having a first end and a second end; the first end configured to couple to a fastening strap for coupling to a first article, and the second end comprising a tapered tab configured to feed the feed tab through a loop for coupling to a second article.

Aspect 72. The tab of Aspect 71, wherein the tapered tab is rigid or resilient.

Aspect 73. The tab of Aspect 71 or 72, wherein the tapered tab is curved upwardly from the body.

Aspect 74. The tab of any one of Aspects 71 to 73, wherein the body is rigid or resilient.

Aspect 75. The tab of any one of Aspects 71 to 74, wherein the first end of the body is further configured to couple to, or be integral to a first part of a two-part cooperating fastener for releasably fastening the strap to the loop to secure the first article to the second article when the strap comprises a second part of the cooperating fastener.

Aspect 76. The tab of Aspect 75, wherein the two-part cooperating fastener is a snap, a hook and loop fastener, or an adhesive fastener.

Aspect 77. The tab of Aspect 76, wherein the fastener is a snap and the feed tab is integral to the snap.

Aspect 78. A loop strip, comprising: a loop coupled to a base, the loop configured to receive a fastening strap coupled to a first article, the base having a first side to which the loop is coupled and a second side configured to couple to a second article.

Aspect 79. The strip of Aspect 78, wherein the loop is substantially centered on the base.

Aspect 80. The strip of Aspect 78 or 79, wherein the base is flexible.

Aspect 81. The strip of any one of Aspects 78 to 80, wherein the loop is flexible.

Aspect 82. The strip of any one of Aspects 78 to 81, wherein the loop strip comprises at least two loops.

Aspect 83. The strip of Aspect 82, wherein the at least two loops are substantially aligned and centered along a length of the first side of the base.

Aspect 84. The strip of Aspect 82 or 83, wherein the base comprises a perforation guide for indicating where to cut the loop strip.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. An artificial breast comprising a shell and a filler removably disposed within the shell, said artificial breast comprising non-synthetic material; said artificial breast being shaped and configured to fit securely within a clothing article by compression between a wearer and the clothing article to inhibit movement of the artificial breast relative to the clothing article; wherein:
the shell comprises a distal side defining a curved bra cup and a proximal side configured to conform to contours of a body and configured to expand, the distal side of the shell comprising an inner felt layer configured to form the bra cup and an outer cloth layer for facing the clothing article when worn, and
the filler is removably disposed within the shell in an amount configured to provide form and firmness to the artificial breast and in an amount adequate to cause compression of the artificial breast when worn;
wherein the proximal side of the shell defines a flexible opening for disposing the filler within the shell, and said shell flexibly accommodates different amounts of filler.

2. The artificial breast of claim 1, wherein the non-synthetic material has anti-bacterial, hypoallergenic, moisture wicking, biodegradable, breathable, or thermal regulating properties, or a combination thereof, wherein:
the felt layer of the shell comprises felted Alpaca fiber, wool fiber, rayon fiber, or a combination thereof; and
the filler comprises Alpaca fiber, hemp fiber, wool fiber, cotton fiber, bamboo fiber, silk fiber, rayon fiber, or a combination thereof.

3. The artificial breast of claim 1, wherein the shell further comprises an inner cloth layer comprises Alpaca fiber, hemp fiber, wool fiber, cotton fiber, bamboo fiber, silk fiber, rayon fiber, or a combination thereof.

4. The artificial breast of claim 1, wherein the proximal side of the shell is configured to expand relative to the amount of filler disposed within the shell to conform the proximal side of the shell to the contours of a body.

5. The artificial breast of claim 1, wherein the filler comprises Alpaca fiber.

6. The artificial breast of claim 1, wherein the inner felt layer of the shell comprises felted Alpaca fiber.

7. The artificial breast of claim 1, wherein the proximal side of the shell comprises bamboo and/or cotton.

8. The artificial breast of claim 1, wherein the distal side of the shell is formed as a horizontally approximately oblong shape comprising an upper portion configured as a semi-oblong, and two lower oblong quadrants forming the lower portion of the oblong shape with an approximately central vertical seam between the quadrants to define the contours of the bra cup.

9. The artificial breast of claim 8, wherein the upper portion of the oblong shape of the distal side of the shell is configured to the wearer's desired shape and volume of bra cup.

10. The artificial breast of claim 8, wherein the upper portion and the two lower portions of the oblong shape of the distal side of the shell are each configured to the wearer's desired shape and volume of bra cup.

11. The artificial breast of claim 1, wherein the bra cup size and shape is suited to the wearer who has had a full mastectomy, a partial mastectomy, and/or who wishes to supplement existing breast volume.

12. The artificial breast of claim 1, wherein the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn.

13. The artificial breast of claim 8, wherein the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn.

14. The artificial breast of claim 1, further comprises a weight disposed within the shell.

15. The artificial breast of claim 14, wherein the weight is a porous rock.

16. The artificial breast of claim 1, wherein the clothing article within which the artificial breast is to be worn comprises a top, a bra, a top, a dress, lingerie, a bralette, or a swimsuit.

17. The artificial breast of claim 1, further comprising at least two fastening straps for securing the artificial breast within a clothing article having loops configured to receive said fastening straps, wherein said fastening straps comprise a ribbon having a first end and a second end, the first end coupling to the shell of the artificial breast; and the second end comprising a feed tab and a fastener, the feed tab being configured for feeding the second end of each of the fastening straps through the respective loop of the clothing article.

18. The artificial breast of claim 17, wherein the fastener is a snap, a hook and loop fastener, or an adhesive fastener.

19. The artificial breast of claim 17, wherein the filler comprises Alpaca fiber in a loose or spun form, and the inner felt layer of the shell comprises felted Alpaca fiber, wherein the contacting of the Alpaca fiber in loose or spun form with the felted Alpaca fiber causes friction fit within the artificial breast to keep filler in position when worn.

20. The artificial breast of claim 17, wherein the distal side of the shell is formed as a horizontally approximately oblong shape comprising an upper portion configured as a semi-oblong, and two lower oblong quadrants forming the lower portion of the oblong shape with an approximately central vertical seam between the quadrants to define the contours of the bra cup.
